(19) United States
(12) Plant Patent Application Publication
(10) Pub. No.: US 2002/0194662 P1
(43) Pub. Date: Dec. 19, 2002

(54) Chrysanthemum plant named 'GEDI ONE SOR'

(76) Inventor: Dirk Pieters, Oostnieuwkerke (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,958

(22) Filed: Jun. 4, 2001

(51) Int. Cl. A01H 5/00

(52) U.S. Cl. PLT/289

(57) ABSTRACT

A new and distinct Chrysanthemum plant variety characterized by a large decorative type inflorescence, consistent flowering response to short days, blooming consistently after 49 days of short day length, free branching habit, and consistent natural season flowering habit during the first two weeks of September.

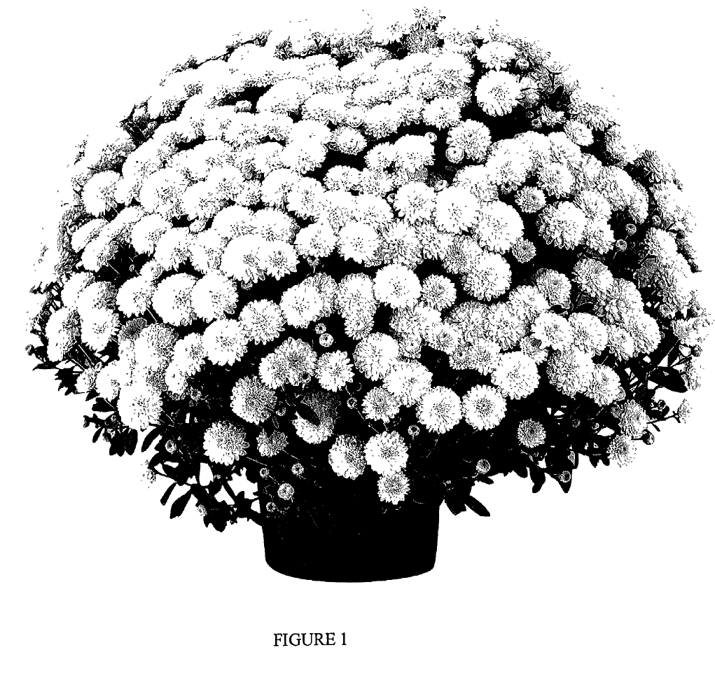

### BACKGROUND OF THE INVENTION

The present invention relates to a new and distinct cultivar of Chrysanthemum plant, botanically known as Dendranthema morifolium and herein after referred to by the cultivar name ‘GEDI ONE SOR.’

This new cultivar originated from hybridization of a female parent, cultivar ‘Dark Veria’ (U.S. Plant Pat. No. 10,216), and a male parent, cultivar ‘Orka’ (unpatented in the United States). The new cultivar was discovered by the inventor in September 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE SOR’ has not been observed under all possible environmental conditions. The phenotype may vary somewhat with variations in environment such as temperature, daylength, and light intensity, without, however, any variance in genotype.

The following traits have been repeatedly observe and are determined to be the unique characteristics of ‘GEDI ONE SOR.’ These characteristics in combination distinguish ‘GEDI ONE SOR’ as a new and distinct Chrysanthemum:

1. Large decorative type inflorescence;

2. Consistent flowering response to short days, blooming consistently after 49 days of short day length;

3. Free branching habit; and

4. Consistent natural season flowering habit during the first two weeks of September.

Plants of the new cultivar are similar to plants of the parent cultivar, ‘Dark Veria,’ in most horticultural characteristics, however plants of the new cultivar differed from plants of the cultivar ‘Dark Veria’ in the following characteristics:

1. The new cultivar has a wider diameter flower;

2. The new cultivar has many more ray florets than the parent;

3. The new cultivar is taller and wider; and

4. The new cultivar naturally blooms 3 weeks earlier.

Plants of the new cultivar are also similar to plants of the parent cultivar, ‘Orka,’ in most horticultural characteristics, however plants of the new cultivar differed from plants of the cultivar ‘Orka’ in the following characteristics:

1. The new cultivar has a wider diameter flower;

2. The new cultivar has many more ray florets than the parent; and

3. The new cultivar is taller and wider.

The new cultivar can be compared to the Chrysanthemum cultivar ‘CLEAGAR GF1’ (disclosed in U.S. Plant Pat. No. 10,463). However plants of the new Chrysanthemum differed from the plants of the cultivar ‘CLEAGAR GF1’ in the following characteristics:

1. The new cultivar naturally blooms 2 weeks earlier;

2. The new cultivar has larger diameter blooms;

3. The new cultivar has more blooms per flowering branch; and

4. The new cultivar is taller and wider.

### BREIF DESCRIPTION OF THE PHOTOGRAPHS

The photograph shows a plant of ‘GEDI ONE SOR’ grown in a 2-gallon container. A single cutting was used in the pot, planted mid-May and grown under outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE SOR’

Commercial classification: Garden-type Chrysanthemum

Parentage: Hybridization of female parent ‘Dark Veria’ (U.S. Plant Pat. No. 10, 216) and male parent ‘Orka’ (unpatented in the United States)

### PROPAGATION

Time to Rooting: 7 to 14 days at approximately 21° C.

Rooting habit: Fine, fibrous

### PLANT

Growth Habit: Mounding herbaceous perennial

Height: Approximately 27 cm

Growth rate: Moderate

Branching Characteristics: Free Branching

Length of Lateral Branches: Approximately 21 cm

Number of leaves per lateral branch: Approximately 22

Stem color: Near 138A

### FOLIAGE

Leaf:

Arrangement.—Alternate single.

Average length.—Approximately 6.2 cm.

Average width.—Approximately 3.0 cm.

Shape of blade.—Ovate.

Apex.—Acuminate.

Base.—Attenuate.

Attachment.—Stalked.

Margin.—Serrate.

Texture of top surface.—Lightly pubescent.

Texture of bottom surface.—Lightly pubescent.

Color.—young foliage upper side: Near 137A young foliage under side: Near 137B mature foliage upper side: Near 137A mature foliage under side: Near 137B venation upper side: Near 138C venation under side: Near 137C.

### Petiole:

Average length.—Approximately 2.1 cm.

### BLOOM

Inflorescence:

Flowering habit.—Induced by darkness period greater than 13.5 hours, approximately 49 days of appropriate day length required to induce and develop blooms.

Inflorescence form.—Decorative.

Natural flowering season.—Approximately the first two weeks of September.

Number of inflorescences per stem.—Approximately 16.

Inflorescence diameter.—Approximately 5.5 cm.

Inflorescence depth.—2.5 cm.

Inflorescence longevity on plant.—Approximately 25-29 days.

Persistence.—Persistent.

Ray Florets:

Appearance.—Matte.

Texture.—Smooth.

Average number per flower.—300.

Shape.—Oblanceolate.

Margin.—Entire.

Apex.—Obtuse.

Length.—Approximately 3.1 cm.

Width.—Approximately 0.8 cm.

Color.—upper surface at first opening: Near 13B upper surface at maturity: Near 13B upper surface at fading: Near 13C under surface at first opening: Near 13B under surface at maturity: Near 13B under surface at fading: Near 13C.

Disc florets:

Appearance.—Shiny.

Texture.—Smooth.

Average number per flower.—Approximately 20.

Shape.—Cylindric.

Apex.—Obtuse.

Average length.—Approximately 1.0 cm.

Average width.—Approximately 0.2 cm.

Color.—at first opening: Near 2A at maturity: Near 7A at fading: Near 16A.

Peduncle:

Length.—at terminal end (shortest): Approximately 5 cm at lateral end (longest): Approximately 11 cm.

Strength.—Moderate.

Color.—Near 137C.

Habit.—Erect.

Inflorescence bud:

Length.—Approximately 1.0 cm.

Diameter.—Approximately 1.0 cm.

Form.—Globular.

Color.—Near 13A.

Sepals:

Appearance.—Matte.

Texture.—Lightly pubescent.

Number.—Approximately 20 to 30.

Shape.—Ovate.

Margin.—Entire.

Apex.—Acute.

Color.—upper side: Near 138A under side: Near 138A.

### REPRODUCTIVE ORGANS

Ray Florets:

Pistillate.—Number of pistils per flower: 1 Stigma shape: 2 branched Stigma color: Near 13B Style color: Near 13D Style length: Approximately 0.6 cm.

Disc Florets:

Pistillate.—Number of pistils per flower: 1 Stigma shape: Cylindric Style length: Approximately 0.5 cm.

Staminate.—Number of stamens per flower: Approximately 5 Anther shape: Tubular Anther color: Near 14C Pollen color: Near 14B.

### OTHER CHARACTERISTICS

Disease Resistance: Neither resistance nor susceptibility observed

### ‘GEDI ONE SOR’: Table of R.H.S. Color Codes

| | | | |
|---|---|---|---|
| FOLIAGE | Leaf | young foliage upper surface | Near 137A |
| | | young foliage under surface | Near 137B |
| | | mature foliage upper surface | Near 137A |
| | | mature foliage under surface | Near 137B |
| | | venation upper surface | Near 138C |
| | | venation under surface | Near 137C |
| BLOOM | Ray Florets | upper surface at first opening | Near 13B |
| | | upper surface at maturity | Near 13B |
| | | upper surface at fading | Near 13C |
| | | under surface at first opening | Near 13B |
| | | under surface at maturity | Near 13B |
| | | under surface at fading | Near 13C |
| | | Pistillate: Stigma | Near 13B |
| | | Pistillate: Style | Near 13D |
| | Disc Florets | at first opening | Near 2A |
| | | at maturity | Near 7A |
| | | at fading | Near 16A |
| | | Staminate: Anther | Near 14C |
| | | Staminate: Pollen | Near 14B |
| | Peduncle | | Near 137C |
| | Inflorescence | | Near 13A |
| | Bud | | |
| | Sepals | upper surface | Near 138A |
| | | under surface | Near 138A |

What is claimed is:

1. A new and distinct variety of Chrysanthemum plant named ‘GEDI ONE SOR’ as herein illustrated and described.
